(19) United States
(12) Patent Application Publication
(10) Pub. No.: US 2009/0112332 A1
(43) Pub. Date: Apr. 30, 2009

(54) BONE GRAFT AND BONE GRAFT SUBSTITUTES WITH ANTIBIOTICS FOR SUSTAINED, LOCALIZED RELEASE OF ANTIBIOTICS FOR REDUCING POSTOPERATIVE SURGICAL WOUND INFECTION IN SPINAL AND OTHER BONE SURGERY

(76) Inventor: Alexis Paul Shelokov, Dallas, TX (US)

Correspondence Address: STORM LLP, BANK OF AMERICA PLAZA, 901 MAIN STREET, SUITE 7100, DALLAS, TX, 75202, US

(21) Appl. No.: 11/932,299

(22) Filed: Oct. 31, 2007

(51) Int. Cl. A61F 2/02; A61F 2/28

(52) U.S. Cl. 623/23.72; 623/16.11

(57) ABSTRACT

A bone graft or bone graft substitute material containing antibiotics is formed into crumbs, powder, putty, flexible sheets, or other form for localized delivery of drugs. The material is adapted to be placed inside a body for sustained, localized delivery of antibiotics. The antibiotics may be in a crystalline or non-crystalline form.

### FIELD OF THE INVENTION

The invention relates to antibiotics delivery systems and methods. More specifically, the invention relates to bone graft and bone graft substitutes with antibiotics for sustained release of local antibiotics for reducing postoperative surgical would infection in spinal and other bone surgery.

### BACKGROUND OF THE INVENTION

In spinal and other bone surgeries, bone grafts and bone graft substitutes are transplanted into the spine, bone or inside the wound cavity to aid in healing or bone formation. One of the most common uses of bone grafts and bone graft substitutes in spine surgery is during spinal fusion. Spinal fusions are performed to relieve pain and provide stability in people who have experienced a vertebral fracture causing pain or spinal deformity. In certain types of spinal fusion, bone grafts or bone graft substitutes are used to replace the cushioning disc material that lies between the vertebrae. When the bone graft or bone graft substitute is placed between the vertebrae, it creates a framework and support that eventually aids in joining the two bones together.

Bone grafts transplanted from one area of a patient into another of the same patient are called bone autografts. The bone graft is harvested from the bones of the hip, the ribs or the leg. Autograft has a low risk of disease transmission and increased likelihood of acceptance in the transplant site. Bone grafts from a donor are called allograft bone. Allograft bone usually comes from bone banks that harvest the bone from cadavers. The types of allograft bone used for spine surgery include fresh frozen and freeze dried. The bone is cleaned and disinfected to reduce the possibility of disease transmission from donor to recipient. Like autograft bone, allograft bone provides a framework for the new bone to grow on and into. The advantages of allograft bone are the elimination of the harvesting surgical site, the related postoperative pain and the added expense of a second operative procedure. Disadvantages of allograft bone are the possibility of disease transmission and lower effectiveness since the bone growth cells and proteins are removed during the cleaning and disinfecting process.

Since autograft and allograft bones have drawbacks, scientists have developed bone graft substitutes that are used in place of autograft and allograft bones. Ceramics have been used as bone graft substitutes. Ceramics do not carry any risk of disease transmission, and are available in many forms such as porous and mesh. Ceramics provide a framework for bone growth, but they do not contain any of the natural proteins that influence bone growth.

Coral have been used as bone graft substitutes. Coral have also been used in spinal surgery as a graft additive, extender, or as a framework for bone to grow into.

Bone Morphogenetic Proteins (BMPs) are a group of growth factors and cytokines known for their ability to induce the formation of bone and cartilage. BMPs are produced in human bodies and regulate bone formation and healing. BMPs have also been used as bone graft substitutes. BMPs are also extracted from humans or cow bones and are also produced in the laboratory.

It has been observed that postoperative infection occurs in 2% to 6% of all surgical cases. Use of bone grafts, instrumentations and prosthesis devices has increased infection rates. In particular, use of instrumentations in spinal surgery has increased infection rates several fold. The average cost of treatment of post operative spine infection (e.g., infections after scoliosis surgery) is estimated to be approximately $150,000.

In management of postoperative spinal and other bone infections, exploration of the wound is an essential first step. The wound is opened in the operating room, and aggressive and often repeated debridement of necrotic tissue and irrigation is performed. Additional bone graft or bone graft substitutes may be implanted inside the wound cavity. At a later time, spinal implants may need to be removed and replaced with new ones. Oral or intravenous antibiotics may also be administered. A course of antibiotics that varies over a period of 4 weeks and 6 months is usually given to reduce the likelihood of infection. While systemically administered antibiotics have been relatively successful in reducing acute infection, it has not been so successful in reducing delayed infections.

### SUMMARY OF THE INVENTION

This invention, in one embodiment, is directed to an implantable, flexible sheet made from one or more bone grafts or bone graft substitutes. The sheet contains a predetermined quantity of one or more selected antibiotics. The sheet is adapted to be placed inside a body for sustained, localized delivery of antibiotics. The bone graft substitute may be a bone morphogenetic protein (BMP), a tri-calcium salt, a demineralized bone matrix or other types of bone graft substitute. The antibiotics may be in a crystalline or non-crystalline form. The flexible sheet may be wrapped about an implant or to be placed inside a wound cavity. In another aspect, the disclosure relates to a putty made from bone graft containing a predetermined quantity of one or more selected antibiotics. The putty is adapted to be placed inside a body for sustained, localized delivery of antibiotics.

### BRIEF DESCRIPTION OF THE DRAWINGS

For a more complete understanding of the features and advantages of the present invention, reference is now made to the detailed description of the invention along with the accompanying figures and in which:

FIG. 1 illustrates a sheet of bone graft or bone graft substitute containing one or more selected antibiotics for sustained, localized delivery of antibiotics.

FIG. 2 illustrates a laminate of bone graft or bone graft substitute containing one or more selected antibiotics for sustained, localized delivery of antibiotics.

FIG. 3A illustrates crumbs of bone graft or bone graft substitute material that contains antibiotics.

FIG. 3B illustrates a crumb of bone graft or bone graft substitute material that contains antibiotics.

FIG. 4A illustrates a putty (or a moldable material) formed with BMP and one or more antibiotics.

FIG. 4B shows an injection used to deliver the putty or the moldable material.

### DETAILED DESCRIPTION OF THE INVENTION

In one embodiment, one or more selected antibiotics are added to bone grafts and/or bone graft substitutes and rolled into a flexible sheet or formed into powder, putty, crumbs or other forms. In another embodiment, bone grafts and/or bone graft substitutes that already contain antibiotics are manufactured into various shapes and forms, including, for example, flexible sheets or as powder, putty or crumbs. The bone graft materials may be autograft or allograft. The bone graft substitutes may be BMPs, tri-calcium salts, demineralized bone matrix (DBM) or ceramics. In this detailed description, the terms “mixed”, “added”, “fortified” and “impregnated” are used interchangeably.

During surgery, the flexible sheet is cut to appropriate size and placed inside a wound cavity. The sheet may also be wrapped or formed about an orthopedic implant, a bone, a tissue or be placed in any part inside a body. The crumbs or putty may also be placed inside a wound cavity or in any other area. The bone graft materials may be autograft or allograft. The bone graft substitutes may be BMPs, tri-calcium salts, demineralized bone matrix (DBM) or ceramics.

In another embodiment antibiotics may be covalently or non-covalently bound with bone grafts (e.g., autograft or allograft) or bone graft substitutes (e.g., BMP, tri-calcium salts, demineralized bone matrix or ceramics) and rolled in sheets or formed into powder, putty or crumbs. The sheets, powder, putty or crumbs may be placed inside a wound cavity or any other area for sustained localized delivery of antibiotics.

As will be understood by those skilled in the art, the bone grafts and bone graft substitutes are transplanted into the spine, bone or inside the wound cavity, or in any other area inside a human body to aid in healing or bone formation. A common use of bone grafts and bone graft substitutes in spine surgery is during spinal fusion. Spinal fusions are performed to relieve pain and provide stability in people who have experienced a vertebral fracture causing pain or spinal deformity. In certain types of spinal fusion, bone grafts or bone graft substitutes are used to replace the cushioning disc material that lies between the vertebrae. When the bone graft or bone graft substitute is placed between the vertebrae, it creates a framework and support that eventually aids in joining the two bones together. Bone graft or bone graft substitutes may also be used in the axial skeleton after trauma, fracture or degeneration of boney body parts such as but not limited to the tibia, femur, wrist, skull and tooth to achieve increased strength, fusion, integrity or pain relief.

The antibiotic is released from the crumbs, powder, putty or sheet into the wound over a period of time. Thus, the crumbs, powder, putty or sheets provide a sustained release of local antibiotics into the wound. The sheets also provide sustained release of local antibiotics with minimal volume effect.

In one embodiment, bone grafts and/or bone graft substitutes are mixed with crystalline antibiotics and rolled into a thin flexible sheet. In another embodiment, bone grafts and/or bone graft substitutes having crystalline antibiotics can be manufactured into various shapes or forms such as, for example rolled into flexible sheets or formed into crumbs, powder or putty. A flexible laminate can be made by bonding a plurality of layers, i.e., sheets. For example, depending on the size of the antibiotics crystals and the number of layers in the flexible laminate, the antibiotics elute out into the wound over a desired period of time. Thus, the flexible laminate having a plurality of layers can operate as a systemic drug delivery system for localized delivery of antibiotics over a period of time. In another embodiment, the antibiotics may be in a non-crystalline form such as a powder, putty or any other suitable form. In another embodiment, a flexible laminate can be made by bonding one or more layers that contain antibiotics and one or more layers that do not contain antibiotics. In other words, a flexible laminate can be made by bonding one or more layers of bone grafts and/or bone graft substitutes containing antibiotics and one or more layers of bone grafts and/or bone graft substitutes not containing antibiotics.

The inclusion of antibiotics in the bone graft or bone graft substitutes will decrease the rate of early and late infection. Also, since sustained delivery of localized antibiotics will reduce infection, fusion and healing will be enhanced.

In posterior or anterior spinal fusion, implants are applied to the spine. Then, antibiotics fortified bone graft materials (e.g., bone graft and/or bone graft substitutes) can be applied to the surface of the bones where fusion is desired. Since some bone graft materials are osteoinductive, bone formation is induced. The inclusion of antibiotics in these bone graft materials will limit the potential development of infection, and decrease the likelihood of future surgery. In one embodiment, bone grafts and/or bone graft materials are mixed with Rifampin and/or Minocycline to decrease acute purulent infection and osteomyelitis.

As discussed before, postoperative infections in orthopedic implants frequently require a patient to undergo a subsequent operation to remove the implant and to sterilize the wound. The flexible sheet described above is intended to reduce the potential for postoperative infection and also prevent the increase in costs resulting from additional procedures necessary to treat the infection.

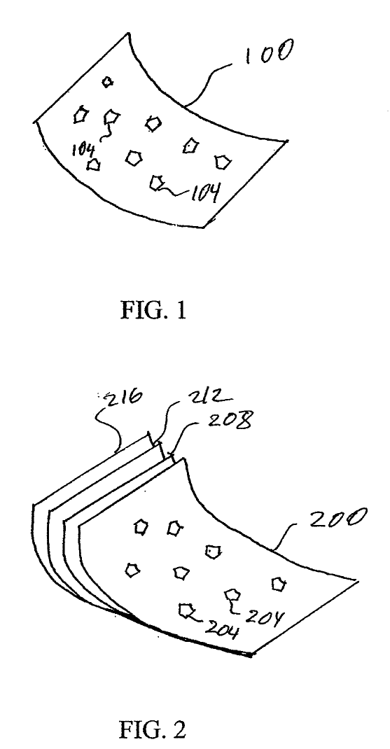

FIG. 1 illustrates a sheet 100 of bone graft and/or bone graft substitutes that contains antibiotics 104. FIG. 2 illustrates a laminate 200 comprising a plurality of layers (e.g., sheets) of bone graft and/or bone graft substitutes that contain antibiotics 204. Individual layers 208-216 in the laminate can be impregnated or mixed with selected types of antibiotics or combination of antibiotics. Thus, individual layers in the laminate can have different types of drugs. Also, individual layers in the laminate can have different absorption characteristics so that different types of drugs can be delivered in controlled manner over a period of time. It will be appreciated that the laminate 200 can be formed by bonding any number of layers. For example, the laminate 210 may have one or more layers that contain antibiotics but other layers that do not contain antibiotics. As will be understood by those skilled in the art, by having only some of the layers impregnated with antibiotics (e.g., alternate layers impregnated with antibiotics), further control can be achieved in the sustained delivery of localized antibiotics. Also, by varying the thickness of the layers and/or varying the amount or size of the antibiotics crystals, further control can be achieved in the sustained delivery of localized antibiotics.

The approximate thickness of a laminate (or sheet) can be from hundredths of a millimeter to several millimeters. It will be appreciated by those skilled in the art that laminates or sheets with other thickness can also be made. Antibiotic crystals can have various sizes (e.g., micron or submicron magnitude). The laminates or sheets may also be fortified with dissolved or powdered antibiotics.

In one embodiment, a method for localized delivery of antibiotics inside or near a wound, a wound cavity or about an implant or any other part inside a body includes wrapping (or forming about) the implant (or any other area inside the body) with one or more sheets of antibiotics fortified bone graft and/or bone graft substitutes. In another embodiment, a method for localized delivery of antibiotics inside or near a wound, a wound cavity or about an implant or any other part inside a body includes wrapping (or forming about) the implant (or any other area inside the body) with one or more cellophanes made from bone graft and/or bone graft substitute or bone graft and/or bone graft substitute putty containing antibiotics.

For example, cellophane laminates may be made from bone graft and/or bone graft substitutes having one or more types of selected antibiotics, thereby creating a multiple layered structure of bone graft or bone graft substitutes that can also deliver localized antibiotics. During or prior to surgery, these layered materials are cut into appropriate size and placed inside a wound cavity or in any other area inside the body. The thin size of the cellophanes or films do not impede the process of implantation.

As will be understood by those skilled in the art, when used intra-venously (IV), antibiotics penetrate hard tissue relatively poorly. The drug delivery system comprising sheets or putty of bone graft or bone graft substitute materials allow sustained localized delivery of antibiotics directly to the hard tissue (e.g., bone) to increase the penetration rate of the antibiotics. As a result, the drug delivery system decreases post operative infection rate.

In one embodiment, a drug delivery system comprises a flexible laminate of multilayered, bone graft materials (e.g., bone grafts and/or bone graft substitutes such as BMPs), wherein at least one layer is impregnated or mixed with a combination of two or more crystalline antibiotics. For example, a combination of Vancomycin and Tobramycin, which is known to be locally effective against Staphylococcal and Streptococcal species, can be delivered using the flexible laminate. In another embodiment, each layer of the flexible laminate can be impregnated or mixed with a particular type of antibiotics or a particular combination of antibiotics. For example, the first layer can be impregnated with a first type of antibiotic and the second layer can be impregnated with a second type of antibiotics.

The amount of time over which the antibiotics elute out, also referred to herein as the absorption time, can be increased by increasing the number of layers in the flexible laminate and also by increasing the size of the crystalline or non-crystalline particles of antibiotics. The elution time or the absorption time, accordingly, can be decreased by decreasing the number of layers in the laminate and also by decreasing the size of the crystalline or non-crystalline antibiotics. The elution time can also be controlled by the relative concentration of the antibiotics in the bone grafts and/or bone graft substitutes during manufacture. The elution time can also be controlled by the porousity of the putty.

For example, the critical time for inhibition of bacteria delivered inadvertently to the wound during a surgery may be two to five days. The number of layers in the laminate and the particle size of the crystalline or non-crystalline antibiotics can be adjusted appropriately so that the antibiotics in combination or alone (e.g., Tobramycin, Vancomycin or a combination of the two) elute out to the wound over a period of two to five days.

In another embodiment, bone graft materials (e.g., bone grafts and/or bone graft substitutes such as BMPs) are used as a carriers for antibiotics. In one embodiment, bone graft materials are impregnated or mixed with antibiotics and fabricated as crumbs. The antibiotics may be in a crystalline or any other suitable form. The antibiotics impregnated crumbs are then introduced to in the wound cavity or any other area for localized delivery of antibiotics. The antibiotics impregnated crumbs may be used in an initial surgery for localized delivery of antibiotics to reduce the possibility of postoperative infection or during a surgery to treat postoperative infection.

For example, a patient with a postoperative infection can be returned to the operating room where aggressive debridement of all necrotic tissue and irrigation is performed. The antibiotic impregnated crumbs are then introduced into the wound cavity. The antibiotics are eluted over a number of days determined by the characteristics of the crumbs and the crystalline antibiotics. In a spinal surgery, the antibiotics impregnated crumbs or putty promote healing, resolution of the infection and fusion of the spine. Accordingly, the dual aim of delivering localized antibiotics and achieving spinal fusion can be met.

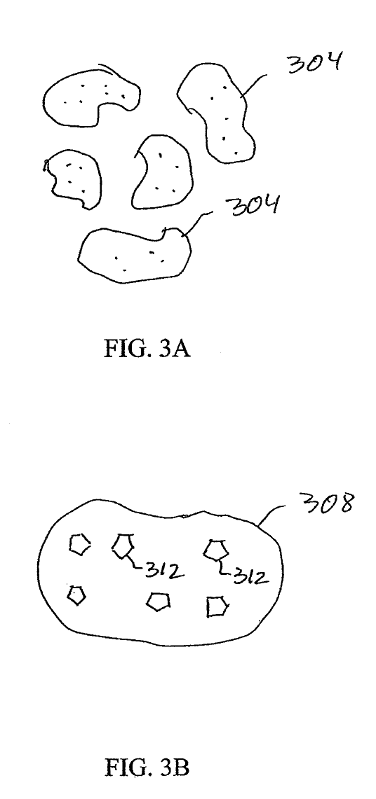

FIG. 3A illustrates crumbs 304 of bone grafts and/or bone graft substitutes that contains antibiotics. The bone grafts may be allograft or autograft, and the bone graft substitutes may be BMPs, ceramics or other substitutes. FIG. 3B illustrates a crumb 308 of bioabsorbable implantable material that is impregnated with antibiotics 312.

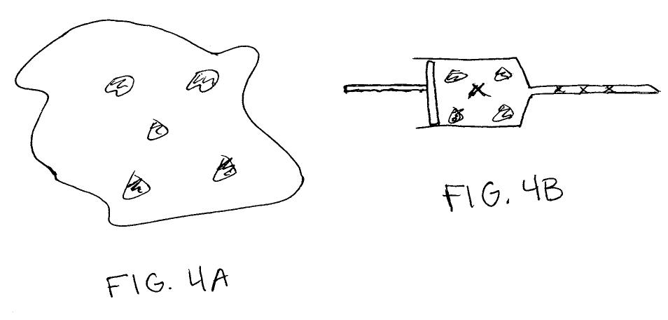

FIG. 4A illustrates a putty (or a moldable material) formed with BMP and one or more antibiotics. FIG. 4B shows an injection used to deliver the putty or the moldable material. An advantage of using the putty or the moldable form of BMP with antibiotics is that the putty (or the moldable material) can conform to the bone and/or the implant (e.g., hardware).

While the compositions, structures, apparatus and methods of this invention have been described in terms of preferred embodiments, it will be apparent to those of skill in the art that variations may be applied to the compositions, structures, apparatus and/or methods and in the steps or in the sequence of steps of the method described herein without departing from the concept, spirit and scope of the invention. All such substitutes and modifications apparent to those skilled in the art are deemed to be within the spirit, scope and concept of the invention as defined by the appended claims.

What is claimed is:

1. An implantable, flexible sheet made from bone graft, the sheet containing a predetermined quantity of one or more selected antibiotics, the sheet adapted to be placed inside a body for sustained, localized delivery of antibiotics.

2. The implantable, flexible sheet of claim 1, wherein the bone graft is bone allograft.

3. The flexible sheet of claim 1, wherein the bone graft is bone autograft.

4. The flexible sheet of claim 1, wherein the antibiotics is in a crystalline form.

5. The flexible sheet of claim 1, wherein the antibiotics is in a non-crystalline form.

6. The flexible sheet of claim 1, wherein the sheet is adapted to be wrapped about an implant.

7. The flexible sheet of claim 1, wherein the sheet is adapted to be placed inside a wound cavity.

8. An implantable, flexible sheet made from one or more bone graft substitutes, the sheet containing a predetermined quantity of one or more selected antibiotics, the sheet adapted to be placed inside a body for sustained, localized delivery of antibiotics.

9. The flexible sheet of claim 8, wherein the bone graft substitute is a bone morphogenetic protein (BMP).

10. The flexible sheet of claim 8, wherein the bone graft substitute is a tri-calcium salt.

11. The flexible sheet of claim 8, wherein the bone graft substitute is a demineralized bone matrix.

12. The flexible sheet of claim 8, wherein the antibiotics is in a crystalline form.

13. The flexible sheet of claim 8, wherein the antibiotics is in a non-crystalline form.

14. The flexible sheet of claim 8, wherein the sheet is adapted to be wrapped about an implant.

15. The flexible sheet of claim 8, wherein the sheet is adapted to be placed inside a wound cavity.

16. An implantable, flexible sheet of cellophane made from one or more bone graft substitutes, the cellophane containing a predetermined quantity of one or more selected antibiotics, the cellophane adapted to be placed inside a body for sustained, localized delivery of antibiotics.

17. The flexible sheet of cellophane of claim 16, wherein the bone graft substitute is a bone morphogenetic protein (BMP).

18. The flexible sheet of cellophane of claim 16, wherein the antibiotics is in a crystalline form.

19. The flexible sheet of cellophane of claim 16, wherein the antibiotics is in a non-crystalline form.

20. The flexible sheet of cellophane of claim 16, wherein the sheet of cellophane is placed inside a wound cavity.

21. The flexible sheet of cellophane of claim 16, wherein the bone graft substitute is a tri-calcium salt.

22. A flexible laminate made by bonding two or more layers of bone graft, at least one of the layers containing a predetermined quantity of one or more selected antibiotics, the flexible laminate adapted to be placed inside a body for sustained, localized delivery of antibiotics.

23. The flexible laminate of claim 22, wherein the bone graft is bone allograft.

24. The flexible laminate of claim 22, wherein the bone graft is bone autograft.

25. The flexible laminate of claim 22, wherein the antibiotics is in a crystalline form.

26. The flexible laminate of claim 22, wherein the antibiotics is in a non-crystalline form.

27. A method for sustained, localized delivery of antibiotics, comprising the step of placing a flexible sheet of bone graft containing a predetermined quantity of one or more selected antibiotics inside a body.

28. A method for sustained, localized delivery of antibiotics, comprising the step of placing a flexible sheet of bone morphogenetic protein containing a predetermined quantity of one or more selected antibiotics inside a body.

29. A putty made from bone graft containing a predetermined quantity of one or more selected antibiotics, the putty adapted to be placed inside a body for sustained, localized delivery of antibiotics.

30. The putty of claim 29, wherein the bone graft is bone allograft.

31. The putty of claim 29, wherein the bone graft is bone autograft.

32. The putty of claim 29, wherein the antibiotics is in a crystalline form.

33. The putty of claim 29, wherein the antibiotics is in a non-crystalline form.

34. A substance made from bone graft containing a predetermined quantity of one or more selected antibiotics, the substance adapted to be placed inside a body for sustained, localized delivery of antibiotics.

35. The substance of claim 34, wherein the bone graft is bone allograft.

36. The substance of claim 34, wherein the bone graft is bone autograft.

37. The substance of claim 34, wherein the antibiotics is in a crystalline form.

38. The substance of claim 34, wherein the antibiotics is in a non-crystalline form.

39. The substance of claim 34, wherein the substance is in a powdered form.

40. The substance of claim 34, wherein the substance is in crumbs form.

41. A substance made from bone graft substitute containing a predetermined quantity of one or more selected antibiotics, the substance adapted to be placed inside a body for sustained, localized delivery of antibiotics.

42. The substance of claim 41, wherein the bone graft substitute is a BMP.

43. The substance of claim 41, wherein the antibiotics is in a crystalline form.

44. The substance of claim 41, wherein the antibiotics is in a non-crystalline form.

45. The substance of claim 41, wherein the substance is in a powdered form.

46. The substance of claim 41, wherein the substance is in crumbs form.

47. The substance of claim 41, wherein the substance is in a putty form.
